(19) United States
(12) Patent Application Publication
(10) Pub. No.: US 2011/0137242 A1
(43) Pub. Date: Jun. 9, 2011

(54) SYSTEM, METHOD AND DEVICE FOR MAKING AND DELIVERING GENETIC-BASED PERSONALIZED NUTRIENT AND SUPPLEMENT MIXTURES

(75) Inventor: Fredric David Abramson, Gaithersburg, MD (US)

(73) Assignee: AlphaGenics, INc., Rockville, MD (US)

(21) Appl. No.: 13/027,427

(22) Filed: Feb. 15, 2011

Related U.S. Application Data
(63) Continuation of application No. 11/459,538, filed on Jul. 24, 2006.
(60) Provisional application No. 60/701,508, filed on Jul. 22, 2005.

(51) Int. Cl. A61M 37/00

(52) U.S. Cl. 604/82

(57) ABSTRACT

A system for preparing a genetically-based personalized mixture of nutrients and other ingredients that includes an input device to input genetic information of a user into the system, an identifying device to identify a genetic profile of the user based on the user input information, and a processing device to process the information inputted by the user and identify a mix and concentration of ingredients stored in the system to be prepared based on the genetic profile of the user. The system also includes a mixing device to mix a customized beverage to deliver to the user where the customized beverage includes the mix and concentration of ingredients prepared based on the genetic profile of the user.

### CROSS-REFERENCE TO RELATED APPLICATIONS

This application claims the benefit of Provisional Application No. 60/701,508, filed Jul. 22, 2005, in the United States Patent and Trademark Office, the disclosure of which is incorporated herein by reference.

### BACKGROUND OF THE INVENTION

1. Field of the Invention

The present invention relates to a system and method capable of using genetic information about an individual to prepare and deliver customized mixtures of nutrients and other supplements, where the choice of ingredients and the amount of each ingredient is adjusted to match the person's genetic profile and lifestyle activity, where the lifestyle activities include sports and physical performance, weight control, and appearing more youthful and the customized mixtures can be delivered in a variety of forms including beverages.

2. Description of the Related Art

At the present time, there are a variety of sports and performance beverages, such as Gatorade® and Powerade,® and others. Sports drink manufacturers emphasize that water is not enough. Gatorade tells consumers that water ‘turns off’ thirst before complete re-hydration occurs. This can even be dangerous for athletes such as marathon runners, due to the fact that an excess of water tends to deplete cells of vital electrolytes. Additionally, carbonated beverages that contain 10 to 11 percent of carbohydrates absorb slowly within the body and do not provide the required immediate energy during physical activity. The carbonation also produces carbon dioxide gas in the stomach and may cause gastrointestinal distress.

There are many sports beverages based on one idea: quench thirst and one-size-fits-all doses of electrolytes and carbohydrates. Most of these sports and performance beverages use standardized formulations so that the brand delivers the same mix to all persons. Their ingredients may include combinations of glucose, pyruvate, branched-chain amino acids, glutamine, arginine, creatine, vitamins, minerals, and caffeine. For example, caffeine may improve athletic performance as a stimulant. However, caffeine also stimulates urine production and contributes to dehydration if taken before exercise. Arginine is an amino acid with no known exercise benefit, and it may cause digestive distress.

Further, there may be differences in the beverage composition from one brand to another in terms of the types of nutrients and their concentration. Most of these beverages deliver energy in the form of carbohydrates, fats and proteins. Many of these products use set levels of electrolytes for replacement and vitamins and nutraceuticals for repair and maintenance. Some brands provide different types and percentages ingredients depending on where in the exercise cycle the person is: pre-exercise, mid-exercise, or post-exercise.

Currently, there is no beverage that is personally tailored to meet a person's genetic and physiological requirements at a given time. There are no beverages that are formulated to match a person's specific genetic profile and the activity in select genes as these influence a person's physical and mental status.

### SUMMARY OF THE INVENTION

The foregoing and other objects of the present invention are achieved by a system for preparing genetic-based personalized mixtures of nutrients that can be conveniently delivered to the consumer in such forms as a beverage. The system includes the identification of genetic information of a person, using that genetic information to identify the selection and concentrations of one or more ingredients, preparing the precise mixture and amounts of the selected ingredients, and delivering that preparation to the person for consumption. The preparation can be delivered in a variety of formats, including ready-to-drink, as a liquid concentrate that can be added to a variety of solids or liquids, as a powder concentrate in loose, capsule or tablet form that can be consumed directly or be added to a variety of solids or liquids, or through a vending system that mixes and prepares the beverage on the spot at the point of sale.

The foregoing and other objects of the present invention are achieved by providing a vending system for preparing a genetic-based personalized beverage. The system includes an input device to input genetic information of a user into the system, an identifying device to identify a genetic profile of the user based on the user input information, a computer programming device to process the information inputted by the user to identify a mix and concentration of ingredients stored in the system, the mix and concentration being prepared based on the genetic profile of the user, and a mixing device to mix a customized beverage to deliver to the user, the customized beverage including the mix and concentration of ingredients prepared based on the genetic profile of the user.

The foregoing and other objects of the present invention are achieved by providing a method for preparing a genetic-based personalized beverage in a vending system. The method includes inputting genetic information of a user into the system, identifying a genetic profile of the user based on the user input information, processing the information inputted by the user to identify a mix and concentration of ingredients stored in the system, the mix and concentration being prepared based on the genetic profile of the user, and mixing a customized beverage to deliver to the user, the customized beverage including the mix and concentration of ingredients prepared based on the genetic profile of the user.

The foregoing and other objects of the present invention are achieved by providing a vending system for preparing a genetic-based personalized beverage. The system includes a mixing unit mixing the beverage, an identifying unit identifying genes and genetic activity of a user, and a transferring unit transferring data about the genes and genetic activity of the user to the mixing unit to mix the beverage, the genes and genetic activity providing a genetic profile, wherein the mixing unit takes a combination and concentration of ingredients based on the genetic profile of the user to prepare and deliver a customized beverage.

### DETAILED DESCRIPTION OF THE PREFERRED EMBODIMENTS

The genetic profile selectively targets genes that are implicated in selected physical, mental, biochemical and/or physiological processes. These include genes involved in the physiology and biochemistry of cells in muscles and nerves, as well as other cells and/or tissues and/or organs that are involved in or related to physical and/or mental activity as well as other lifestyle-related processes. The purpose of the invention is to provide a personalized mixture that improves lifestyle functions including physical and or mental performance, a sustainable level of physical and/or mental activity, and/or recovering from the effects of physical and/or mental performance. An additional purpose of the invention is to deliver a personalized beverage based on a person's genetic profile and activity in selected genes that helps improve desired lifestyle outcomes such as weight control, aging, and skin appearance. Another purpose of the invention is to deliver a personalized beverage based on the person's genetic profile and activity in selected genes that helps confer protection against one or more contagious viral disease. A level of adjustment in the present invention relates to the type of physical activity, the duration of the activity, and the individual effects of the activity. A mix and concentration of nutrients for an individual may vary depending on the type of activity and when in the activity cycle the beverage is consumed. For example, a person planning a heavy workout for one hour may require one beverage configuration if taken before the workout, as the body's relevant processes have not yet started to respond to the activity. Once the activity is started, the mix and concentrations are likely to change in order to deliver a reasonably optimized physical and/or mental performance. After the activity is completed, another mix and with different concentrations may be required to help the person recover. An example of recovery help would be to accelerate the clearance of lactic acid from the muscles. Lactic acid accumulates when the muscles are working, and lactic acid buildup is one limiting factor in sustained performance.

Accordingly, it is an object of the present invention to utilize genetic information about an individual to prepare and deliver customized beverages and other consumables, where the ingredients included in the beverage or consumable and the amount of each ingredient is adjusted to match the person's genetic profile and genetic activity.

Additional aspects and/or advantages of the invention will be set forth in part in the description which follows and, in part, will be apparent from the description, or may be learned by practice of the invention.

A feature of the present invention is not obvious to molecular biologists, nutritionists and others schooled in the art is the need to measure a broad spectrum of genes for each person. This broad spectrum would include genes that are not currently directly implicated in the lifestyle area of concern, such as exercise, physiology, neurology or other areas that relate to physical and/or mental performance. This is because the ongoing scientific research continues to identify genes whose relationship to various lifestyle activities are previously unrecognized. Thus, the methodology includes the use of various technologies that measure gene expression in larger numbers of genes.

According to the present invention, a beverage may be delivered to a user via a custom-blending vending device that prepares the customized beverage at the point of sale. The personalized beverage can also be prepackaged and delivered to the customer in ready-to-drink form in cans, bottles and/or flexible containers. The flexible containers may be re-sealable to that the user can safely carry the personalized beverage without spilling.

According to the present invention, the personalized mixture can also be delivered to the customer in concentrated form as a liquid or solid, which can be taken directly or mixed with other solids or liquids. The solid concentrate can be packaged as a powder, in a capsule or in tablet form.

The present invention provides an individualized, custom-blended beverage whose formula is based on an individual's genetic makeup. It is designed for the sports, physical and mental performance market, to assist with increased performance, weight loss, and for anti-aging.

The beverage of the present invention is a custom-blended combination of key nutrients and other ingredients that take into account a person's physical and mental demands as well as the person's genetic and biochemical makeup. For example, appropriate nutrients and other ingredients that are supplied to the genes involved in biochemical pathways that affect metabolism, such as lactic acid production/degradation as well as ATP generation, may produce better performance outcomes.

According to the present invention, the nutrients and other ingredients supplied to each person are more closely matched to what that person actually needs and from which the person can benefit based on each person's genetic and biochemical profile, activity level, and timing.

The beverage of the present invention configures an appropriate mixture based on the individual's genetic profile. The mixture is based on the presence or absence of different genes associated with targeted biochemical pathways that are stressed during physical activity. The mixture is individually adjusted based on the specific activity and when it takes place. What the body needs before starting exercise can be quite different from what it needs when the exercise is finished.

The application of the beverage of the present invention can be extended to other lifestyles and other health applications. For example, varied ingredient configurations can be matched against a person's genes related to and including metabolism, fat burning, hunger and fat storage in order to help the person not look as old as his/her age. Another application could target genes related to the aging process in order to slow aging or to help the person not look as old as he or she is. Another application would be to prepare nutrient mixtures that help with skin care and skin appearance. These applications and others not enumerated here all use specific genetic information about the individual and provide the appropriate blend and concentration of ingredients to produce the best result. The mixing process can take place using a vending device of the type described herein, or can be performed manually at the point of sale. Additionally, the blend and concentration of ingredients can be added to various types of beverages, such as but not limited to sodas, milk drinks, smoothies and other flavored or unflavored drinks. Additionally, various taste and flavor enhancers, as well as inert ingredients that aid in consistency, stability, shelf life, and other characteristics can be added to the blend and concentration of ingredients to make the final drink more acceptable to the individual's taste.

A specific genotype of genes in a person and, as needed, the activity of one or more of these genes, can be identified using conventional methods including, but not limited to: high density microarrays, PCR, optical reading and biochemical testing, as well as through various questionnaire devices where the respondent's answers can be reasonably correlated with, and therefore predict, the person's underlying genetic makeup and genetic activity profile for the genes in question.

The tissues and bodily fluids that can provide genetic, physiological and/or biochemical information include saliva, cheek cells, blood, skin, hair and other tissues and bodily fluids that can be accessed in a living person.

The beverage of the present invention may also supplement nutrient deficiencies in individuals suffering from one or more health conditions and/or diseases, as well as provide nutrients needed as a result of the person taking one or more medications. For example, people on chemotherapy for cancer often have one or more dietary and nutrient deficiencies. The beverage of the present invention provides a mixture that is based on the person's genetic activity and targets an appropriate mixture for that person's situation. The mixture can also be adjusted over time as the person's genetic profile changes.

Another application of the beverage of the present invention may be used for animal performance. In particular, certain types of animals including race horses, and dogs, and other types of animals are engaged in various activities which require physical endurance and stamina. The appropriate mixture and concentrations of one or more ingredients that are calibrated based on the genes and genetic activity of those genes in the animal can improve the animal's performance. Similarly, mental acuity in the animal can be improved.

Accordingly, the present invention may be implemented in various ways. For example, the present invention may provide a plurality of cylinders. However, the present invention is not limited to cylinders and may use other types of configurations. Each cylinder contains a liquid containing at least one ingredient. The ingredient may be a carbohydrate, a fat or a protein, a mineral or a phytochemical. The phytochemical may be a purified extract from a plant or a plant extract that contains other naturally occurring chemicals.

The cylinders are arrayed on a platform. The cylinders may be placed in a circular fashion around a central core or in a grid arrangement. The contents of each cylinder are delivered to the drink using a pressure mechanism. The pressure mechanism is calibrated in standard ways to deliver a measured dose to the drink. Cylinder contents may be delivered to the drink using a flexible tube that is connected at one end to the cylinder and at the other end to a position that permits the liquid to be delivered to the drink.

The platform may be moveable, so that a selected cylinder is positioned at a point to deliver the cylinder's contents to the drink. The delivery may be delivered through the use of a pressure device or a gravity feed device. Alternatively, the platform is stationary, and a cylinder's contents are delivered to the drink using a flexible tube or other similar conveyance.

The container for the final drink may be a cup or closable container that can be sealed with a screw-type cap or some other covering that prevents the container's contents from spilling. One variation has the contents of final drink container shaken or stirred in such a fashion to create an effervescent or foamy appearance. Another variation has the nutrient in a powder form, where each specific nutrient is delivered to the drink container. The drink container may contain a fluid either before the powder is delivered or afterward, depending on the solubility and mixability of the powders. The drink flavoring may be contained in one or more of the cylinders in a liquid or suspension, or in a powder form.

Further, the present invention may be a tube cleaning mechanism which cleans each tube connected to a cylinder. The tube cleaning is designed to maintain good flow as well as limit the growth of micro-organisms and/or fungi in the tube or cylinder.

The present invention provides a vending system that has one or more data entry mechanisms designed to aid the purchaser in obtaining the best drink. One data entry mechanism may identify the purchaser so that the proper drink configuration can be delivered. Another data entry mechanism may identify the kind of activity the purchaser is or was engaged in. The activity profile may be classified by type (i.e., running, free weights, basketball, sexual intercourse) as well as timing of the drink consumption relative to the activity (i.e., whether the drink is consumed before starting the activity, during the activity, or after the activity is completed).

Additionally, the vending system may be equipped with one or more ways to input measures of a person's physiologic state and/or biochemical activity. The input measures include but are not limited to blood pressure, pulse rate, or electrolyte content of the body. The input devices are designed to be minimally intrusive, and may include one or more variations of a pressure cuff, touchable plate, touchable screen, saliva sample collector which may include a swab or receptacle, and/or a mechanism to collect a blood sample from the finger, ear or other body location.

Further, the present invention provides various entry devices in which a person's genetic profile may be entered: a programmable smart card, where the information may be encrypted or unencrypted; wireless communications between the vending machine and various portable devices (i.e., a cell phone, personal digital assistant, I-pod or other MP3 player, a laptop or desktop computer, or other portable device possessing communications abilities) the Internet; a hard-wired connection to a device that connects to the vending machine; a key-pad with numeric, alphabetic or alphanumeric characters; voice input, which includes direct to the vending machine or through a microphone; and a machine-readable card.

The vending system possesses microprocessor computing capabilities, and computer software that uses the user-data inputted into the vending system to identify the mix and concentrations of ingredients. The final mix may include all or some subset of the available ingredients.

An interior of the vending system may be refrigerated or the ingredient cylinders may be individually cooled as required. The ingredient cylinders may be refillable. They may also be pre-filled with the ingredient with the total cylinder replaced with new ingredient contents as needed.

According to another embodiment of the present invention, the vending system may have data-tracking and communication capabilities. The data tracked includes the frequency and volumes of nutrients per drink and in the aggregate for any interval of time. Data captured includes the identity of the users, their frequency of use, their timing relative to the physical activity, time of day, and other useful parameters. The data may be transmitted via the Internet or any other commercially available communications means.

According to the present invention, input devices, which include the smart card, cell phone and PDA, may also be used as payment mechanisms. The user may be able to add funds to the device through the vending system, the Internet or other commercially available means.

According to the present invention, the system to prepare individualized ingredient portions includes the following components: a means of identifying a person's genes (DNA) and genetic activity (RNA) as these relate to physical activity and mental acuity; a means of transferring data about a person's genes to a device that can mix the customized beverage, and a means of mixing a customized beverage where the combination and concentration of ingredients is determined by taking into account the person's genetic profile. Additional personalization takes place with information about the type and timing of activity in which the person will be, is or was engaged.

The personalized information can be additionally used to produce pre-mixed dry-ingredient packets that can be mixed with a liquid of the person's own choice. Personalized pre-mixed case lots can be produced in an assembly line.

Communication between the vending device and other devices uses means that include wireless, including BlueTooth® and Wi Fi, wire connection and USB connections, as well as any other communication means commercially available, including a touch-pad or key-pad The card held by the user contains the means of storing electronic information, including magnetic stripe and embedded chips. The card may be used to store financial information that lets the person purchase drinks without the use of cash. The electronic information regarding the user's genetic profile may be encrypted using conventional encryption methods or novel methods that are otherwise unavailable commercially.

The genes that may be targeted by beverage of the present invention include one or more genes in the following list, as well as other genes not yet identified as relating to physical and mental performance:

TABLE 1

| GeneCard | Name used in | |
|---|---|---|
| Name | literature | Gene Function |
| VEGF | vascular endothelial | Promote vascular growth and vascular |
| | growth factor | permeability. In skeletal muscle it promotes |
| | | the growth of capillaries as a result of oxygen |
| | | deficient conditions during exercise. |
| | | Expressed more in TypeIIb fibres than other |
| | | kinds of muscle cells after exhaustive |
| | | exercise. |
| UCP3 | Uncoupling protein 3 | Mitochondrial transporter proteins that cause |
| | | protein leaks in the inner membrane of the |
| | | mitochondria. May play a role in adaptive |
| | | thermogenesis and energy expenditure. |
| UCP2 | Uncoupling protein 2 | Mitochondrial transporter proteins that cause |
| | | protein leaks in the inner membrane of the |
| | | mitochondria. This protein is expressed in |
| | | skeletal and heart muscle along with adipose |
| | | tissue. |
| UCP1 | Uncoupling protein 1 | Mitochondrial transporter proteins that cause |
| | | protein leaks in the inner membrane of the |
| | | mitochondria. Only expressed in adipose |
| | | tissue. |
| TTN | Titin | A structural protein used in the A band thick |
| | | filament within the sarcomere. Involved in |
| | | the storage and re-utilization of elastic |
| | | energy. |
| TNF | TNF-a, tumor | A cytokine typically produced by |
| | necrosis factor | macrophages to induce cell death. May help |
| | | regulate skeletal apoptosis. |
| THBS2 | thrombospondin 2 | Adhesive glycoprotein that mediates cell to |
| | | cell and cell to ECM communication. Can |
| | | bind to a variety of ECM proteins Expressed |
| | | higher in endurance trained athletes and is |
| | | found in the connective tissue around skeletal |
| | | muscle and tendons |
| THBS1 | thrombospondin 1 | Adhesive glycoprotein that mediates cell to |
| | | cell and cell to ECM communication. Can |
| | | bind to a variety of ECM proteins Expressed |
| | | higher in endurance trained athletes and is |
| | | found in the connective tissue around skeletal |
| | | muscle and tendons |
| TFB2M | TFB2M | Mitochondrial transcription factor necessary |
| | | for the transcription of mitochondrial DNA. |
| | | Interacts with mitochondrial RNA |
| TFB1M | TFB1M | Mitochondrial transcription factor necessary |
| | | for the transcription of mitochondrial DNA. |
| | | Interacts with mitochondrial RNA |
| TFAM | TFAM | Mitochondrial transcription factor capable of |
| | | activating mitochondrial DNA replication and |
| | | expression. Essential to mitochondrial |
| | | genesis and is capable of unwinding and |
| | | bending DNA. Expression is increased with |
| | | chronic exercise. |
| SOD3 | extra cellular | Destroys radicals produced by cells which are |
| | superoxide | typically harmful to biological systems. This |
| | dismutase 3 | process causes the production of H202 which |
| | | is thought to promote the expression of NO |
| | | syntase |
| SOD1 | Cu/Zn superoxide | Destroys radicals produced by cells which are |
| | dismutase | typically harmful to biological systems. This |
| | | process causes the production of H202 which |
| | | is thought to promote the expression of NO |
| | | syntase |
| SLC2A4 | GLUT 4 | Insulin-regulated facalitive glucose |
| | | transporter. An integral protein which resides |
| | | in vesicles below the plasma membrane and |
| | | will fuse with it in response to insulin to help |
| | | allow glucose from the blood into cells. Daily |
| | | exercise increases GLUT4 expression. |
| SKI | SKI | May play a role in the differentiation of |
| | | skeletal muscle cells |
| SFPTA1 | surfactant, | In the presence of calcium it binds to |
| | pulmonary- | suffacant phosoplipids to promote respiration |
| | associated protein | |
| RAD23A | RAD23A | Gene involved in DNA repair. Upregulated |
| | | after intense endurance exercise |
| PYGM | Glycogen | Involved in glycogen metabolism |
| | phosphorylase | |
| PSMC6 | ATPase 6, 26S | Involved in the degradation of ubiquitinated |
| | protease | proteins. |
| PRKAB1 | AMPK | Responsible for the regulation of both fatty |
| | | acid and cholesterol synthesis. |
| PRKAA2 | AMPK | Responsible for the regulation of both fatty |
| | | acid and cholesterol synthesis. Also acts as a |
| | | stress-sensing protein kinase to shut off bio- |
| | | synthetic pathways when ATP is low |
| PPP3CC | calineurin subunit | Calcium-dependent calmodulin-stimulated |
| | | phosphatase |
| PPP3CB | calineurin subunit | Calcium-dependent calmodulin-stimulated |
| | | phosphatase |
| PPP3AB | calineurin subunit | Calcium-dependent calmodulin-stimulated |
| | | phosphatase |
| PPP2R1 | calineurin subunit | Calcium-dependent calmodulin-stimulated |
| | | phosphatase |
| PPARGC1 | PGC1 | Transcriptional cofactor for seroid and |
| | | nuclear receptors. Can regulate |
| | | mitochondrial genes that regulate |
| | | thermogenesis. Upregulated after exercise |
| PLAT | tPA | A protease which converts plasminogen to |
| | | plasmin which is involved in the break down |
| | | of ECM. This gene is upregulated during |
| | | exercise. |
| PKM2 | pyruvate kinase | Metabolic protein involve in glycolysis |
| PER1 | PER1 | Circadian regulator that may act as a |
| | | transcription factor. |
| PER2 | PER2 | Circadian regulator that may act as a |
| | | transcription factor. In an experiment it was |
| | | found to be upregulated 6 hours after |
| | | exercise |
| PDLIM1 | Elfin | Cytoskeleton protein which may act as an |
| | | adapter to bring other proteins to the |
| | | cytoskeleton |
| PDK4 | pyruvate | A glucose regultory gene which inactivates |
| | dehydrogenase | pyruvate dehydrogenase by phosphorylating |
| | kinase 4 | it. The gene is upregulated by exercise and |
| | | reaches peak levels during recovery |
| NSEP | nuclease sensitive | A DNA repair enzyme and negative regulatory |
| | element binding | factor. Upregulated with exercise |
| | protein 1 | |
| NR1D2 | NR1D2 | Gene involved in the circadian clock binds to |
| | | DNA to inhibit Bmal transcription |
| NOS3 | eNOS, nitrous oxide | Produces NO which relaxes vascular smooth |
| | synthase 3 | muscle. Up regulated by the presense of |
| | | H202 which is thought to be produced as a |
| | | result of exercise. |
| NKIRAS2 | NFkB | A transcription factor that can be activated by |
| | | muscle contraction |
| MYOG | Myogenin | Involved in muscle differentiation. Induces |
| | | fibroblasts to differentiate into myoblasts. |
| MYOD1 | MYOD, myoblast | A developmental transcription factor that |
| | determination | helps fibroblast differentiate into myoblasts. |
| | protein | Thought to promote the development of MHC |
| | | type 2b muscle |
| MYL3 | MLC3, myosin light | Regulatory light chain of myosin |
| | chain ventricular, | |
| | skeletal, slow | |
| MYL2 | MLC2, myosin light | Light chain of myosin which is upregulated in |
| | chain cardiac slow | endurance athletes |
| MYL1 | MLC1, myosin light | Regulatory light chain of myosin |
| | chain, skeletal fast | |
| MYH4 | MyHC 2b myosin | Muscle contraction |
| | heavy chain type 2b | |
| MYH2 | MyHC 2a myosin | Muscle contraction |
| | heavy chain type 2a | |
| MYH1 | MyHC 2x myosin | Muscle contraction |
| | heavy chain type 2x | |
| MYF6 | MRF4, herculin | Involved in muscle differentiation. Induces |
| | | fibroblasts to differentiate into myoblasts. |
| MYF5 | MYF5, mygenic | Involved in muscle differentiation. Induces |
| | factor 5 | fibroblasts to differentiate into myoblasts. |
| MYBPC1 | MyBP-C myosin | Think filament associate protein located in |
| | binding protein C | the crossbridge region of skeletal muscle A |
| | slow type | bands. |
| MYBPC2 | MyBP-C myosin | Think filament associate protein located in |
| | binding protein C | the crossbridge region of skeletal muscle A |
| | fast type | bands. |
| MT-RNR2 | Mitochondrially | Mitochondrial ribosome subunit |
| | coded 16S RNA | |
| MT-ND3 | Mitochondrially | Involved in the oxidative pathway |
| | coded NADH | |
| | dehydronase | |
| MB | Myoglobin | Acts as a reserve supply of oxygen and |
| | | facilitates oxygen movement in muscle cells |
| MAPK1 | mitogen activated | Phosphorylates microtubule-associated |
| | protein kinase | protein-2 (MAP2). Myelin basic protein (MBP), |
| | | and Elk-1; may promote entry in the cell |
| | | cycle. |
| MAP2K1 | MEK1 | Activates ERK1 and ERK2 MAP kinases. |
| LPL | lipoprotein lipase | hydrolysis of triglycerides of circulating, |
| | | chylomicrons and very low density |
| | | lipoproteins (VLDL) |
| LIPE | HSL hormone | hydrolyzes stored triglycerides to free fatty |
| | sensitive lipase | acids |
| LEP | Leptin | Regulates adipose tissue mass |
| JUN | Cjun | Transcription factor that forms AP1 |
| | | transcription complex. Highly upregulated |
| | | after exercise |
| IL6 | Interluekin 6 | Cyrokine with a wide range of biological |
| | | functions. Produced by both adipose and |
| | | skeletal muscle as a result of exercise |
| IGF1 | insulin like growth | Growth hormone which can promote muscle |
| | factor | repair and development |
| HYOU1 | ORP-150 | role in cytoprotective cellular mechanisms |
| | | triggered by oxygen deprivation. |
| HSB1 | HSP27 | Stress resistance and actin organization. |
| HSBA8 | HSC70 | Stress chaperone protein |
| HSBA4 | HSP70 | Heat stress induced protein |
| HSF-2 | heat shock | Transcription factor which promotes heat |
| | transcription factor | shock proteins in response to stress |
| HMOX-1 | HO-1, heme | Oxidative stress gene known to increase as a |
| | oxygenase | result of exercise |
| HK2 | hexokinase 2 | Metabolic gene known to increase following |
| | | exercise |
| HERPUD1 | HERPUD1 | Possible involvement in unfolded protein |
| | | response pathway |
| HDAC9 | HDAC | Deacytelates histones which plays role in |
| | | transcriptional regulation, cell cycle |
| | | progression and developmental events |
| HADHS | HAD, hydroxyacyl- | Oxidation of short fatty acids in |
| | Coenzyme A | mitochondria. |
| | dehydrogenase, | |
| | short chain | |
| HADHA | HAD, hydroxyacyl- | Oxidation of short fatty acids in |
| | Coenzyme A | mitochondria. |
| | dehydrogenase long | |
| | chain | |
| GYS1 | GYS, glycogen | Metabolic gene which is upregulated after |
| | synthase | exercise even after a few days |
| GPX3 | GAD glutathione | Protective enzyme that protects cells from |
| | peroxidase 3 | free radicals and detoxifies H2O2 |
| | (plasma) | |
| GPX1 | GAD glutathione | Protects the hemoglobin in erythrocytes from |
| | peroxidase (cellular) | oxidative breakdown |
| GNAS | GNAS | Transmembrane signal transducer |
| GDF8 | MSTN, myostatin | Negative regulator of muscle growth |
| GAPD | GAPDH | Metabolic gene upregulated after endurance |
| | | exercise |
| FOS | Cfos | Plays a role in regulating the development of |
| | | cells that maintain the skeleton. Interacts |
| | | with cjun to form the AP1 transcription |
| | | complex |
| FLT1 | FLT1 | Receptor for VEGF, VEGFB and PGF |
| CYCS | CYC, cytochrome c | Electron carrier protein, plays role in |
| | | apoptosis |
| CS | citrate synthase | Metabolic protein in the citric acid cycle |
| CRYAB | crystallin alpha B | Stress induced molecular chaperone |
| CRY1 | CRY1 | Circadian clock gene |
| CPT1B | CPT1 (muscle) | Metabolic gene |
| CPT1A | CPT1 (liver) | Metabolic gene |
| CLOCK | CLOCK | Circadian clock gene |
| CKM | creatine kinase | Reversibly catalyzes the transfer of phosphate |
| | | between ATP and creatine phosphate |
| CAV3 | caveolin-3 | Possible scaffolding protein in caveolur |
| | | membrane, interacts with and regulates alpha |
| | | Gprotein subunits. Upregulated during |
| | | exercise |
| CAT | Catalase | Protects cells from the toxic effects of H202 |
| CAMK4 | calcium/calmodulin- | a heat-stable, acidic, calmodulin-binding |
| | dependent protein | protein. |
| | kinase | |
| BCL2 | BCL2 | Suppresses apoptosis and inhibits caspases |
| BAX | BAX | Accelerates cell death by helping with the |
| | | release of cytochrome c activating caspase 3 |
| | | and inhibiting Bcl2 |
| ATF2 | CREB2 | Transcription factor upregulated with |
| | | exercise |
| ARNTL | Bmal2 | Activator of circadian clock proteins |
| AREG | Amphiregulin | Bifunctional growth-modulating glycoprotein |
| ALDOA | Fructose- | Metabolic gene in the glycolysis pathway |
| | bisphosphate | |
| | aldolase A | |
| SLC16A1 | MCT1, | Catalyzes the rapid transport across the |
| | Moncarboxylate | plasma membrane of many |
| | transporter 1 | monocarboxylates such as lactate and |
| | | pyruvate, Typically into slow oxidative muscle |
| ACE | ACE1 | Converts angiotensin I to angiotensin II |
| REN | Rennin | Renin is a highly specific endopeptidase, |
| | | whose only known function is to generate, |
| | | angiotensin I from angiotensinogen in the |
| | | plasma |
| SLC16A3 | MCT4, | Catalyzes the rapid transport across the |
| | Moncarboxylate | plasma membrane of many |
| | transporter 4 | monocarboxylates such as lactate and |
| | | pyruvate. Typically out of fast twitch muscle |
| PPARA | PPAR alpha, | Once activated by a ligand, the receptor binds |
| | peroxisome | to a promoter element in the gene for acyl- |
| | proliferator- | CoA, oxidase and activates its transcription. It |
| | activated receptor | therefore controls the peroxisomal beta- |
| | | oxidation pathway of fatty acids. |
| PDK2 | PDK2, pyruvate | Inactivate PDC by phosphorylation. Helps the |
| | dehydrogenase 2 | body conserve glucose |
| PC | pyruvate | Metabolic protein involved in glucose |
| | carboxylase | production or lipid production from pyruvate |
| | | depending on what tissue the pyruvate is in |
| RXRA | RXR-alpha | Nuclear hormone receptor. Involved in the |
| | | retinoic acid response pathway. Binds 9-cis |
| | | retinoic acid (9C-RA). |
| RXRB | RXR-beta | Nuclear hormone receptor. Involved in the |
| | | retinoic acid response pathway. Binds 9-cis |
| | | retinoic acid (9C-RA). |
| RXRG | RXR-gamma | Nuclear hormone receptor. Involved in the |
| | | retinoic acid response pathway. Binds 9-cis |
| | | retinoic acid (9C-RA). |
| SREBF | SREBP-1c | Transcription factor that Regulates the |
| | | transcription of genes for sterol |
| | | biosynthesis and the LDL receptor gene. |
| FOXO1A | FKHR | Thought to be transcription factor for PKD4 |
| | | and LPL.and is upregulated after exercise |
| FSTL3 | FLRG | Possible inhibiter of myostatin upregulated |
| | | with exercise |
| ACVR2B | activin lib | receptor for myostatin which is |
| | | downregulated after exercise |
| EPO | Erythropoietin | Regulation of erythrocyte differentiation and |
| | | the maintenance of a physiological level of |
| | | circulating erythrocyte mass |
| PPARD | PPAR delta | Receptor involved in regulating genes for |
| | | fatty acid oxidation. Thought to help convert |
| | | type 2 muscle fibers to type 1 |
| TNNI1 | troponin I slow | the thin filament regulatory complex which |
| | | confers calcium-sensitivity to striated muscle |
| | | actomyosin ATPase activity. |
| TNNI2 | troponin I fast | the thin filament regulatory complex which |
| | | confers calcium-sensitivity to striated muscle |
| | | actomyosin ATPase activity. |
| CYTB | cytochrome b | Mitochondrial protein involved in the electron |
| | | transport chain |
| HIF1A | HIF-alpha | DNA transcription factor that is produced in |
| | | response to hypoxic conditions |
| ACADL | LCAD, long-chain | Metabolic protein |
| | acyl-CoA | |
| | dehydrogenase | |
| ABCC9 | SUR | Subunit of ATP-sensitive potassium channels, |
| | | maybe be involve in insulin secretion |
| | | regulation |
| CSRP3 | MLP, LIM protein | Regulator of myogenesis. |
| LDHA | lactate | Metabolic enzyme in the glycolytic pathway |
| | dehydrogenase 3 | |
| HIF1AN | HIF1 inhibitor | Serves as an oxygen sensor by inhibiting |
| | | HIF1A under normal conditions |
| DUSP2 | dual specificity | Regulates mitogenic activity by |
| | phosphatase 2 | dephosphoraylting MAP kinase |
| IL1B | IL-1Beta | Proinflammatory cytokine |

Although a few embodiments of the present invention have been shown and described, it would be appreciated by those skilled in the art that changes may be made in these embodiments without departing from the principles and spirit of the invention, the scope of which is defined in the claims and their equivalents.

What is claimed is:

1. A system for preparing a personalized mix of nutrient and supplement ingredients, comprising: an input device to input genetic information of a user into the system; an identifying device to identify a genetic profile of the user based on the user input information; a processing device to process the information inputted by the user and identify a mix and concentration of ingredients stored in the system to be prepared on the genetic profile of the user; and a
mixing device to mix said customized mix of nutrient and supplement ingredients to deliver to the user, the customized beverage including the mix and concentration of ingredients prepared based on the genetic profile of the user.

2. The system according to claim 1, further comprising: a plurality of containers, each cylinder containing a liquid containing at least one ingredient.

3. The system according to claim 2, further comprising: a pressure mechanism to deliver the contents of each of the cylinders to the beverage, the pressure mechanism being calibrated to deliver a predetermined amount of ingredients to the beverage.

4. The system according to claim 2, further comprising: a plurality of flexible tubes to deliver the contents of each of the cylinders to the beverage, each flexible tube being connected at one end thereof to the respective cylinder and at the other end to a position that permits a liquid to be delivered to the beverage.

5. The system according to claim 4, wherein a tube mechanism is provided to clean each of the flexible tubes connected to the cylinders.

6. The system according to claim 1, wherein the identifying device comprises: a first data entry device to identify the user so that the proper beverage ingredients are delivered; and a second data entry device to identify an activity profile of the user, the activity profile being classified by activity type and a timing of the beverage consumption relative to the activity type.

7. The system according to claim 1, wherein the input device is a programmable smart card, a machine-readable card, a wireless communications device, the internet, a hardwired connection, a key-pad, or voice input.

8. The system according claim 1, further comprising: a data-tracking device to track data including the frequency and volumes of nutrients per beverage; and a data capturing device to capture data including the identity of the users, frequency of use, and timing that is relative to user physical activity and time of day.

9. A system for preparing a physiologically personalized mix of nutrient and supplement ingredients, comprising: an input device to input genetic information of a user into the system; an identifying device to identify a genetic profile of the user based on the user input information; a processing device to process the information inputted by the user and identify a mix and concentration of ingredients stored in the system to be prepared based on the genetic profile of the user; a mixing device to prepare the customized ingredient mixture where the mix and concentration of ingredients is prepared based on the genetic profile of the user, and a packaging device to deliver the customized ingredients to the user as a beverage, liquid concentrate, powder concentrate, capsule or tablet.
